(19) United States
(12) Patent Application Publication
(10) Pub. No.: US 2006/0127962 A1
(43) Pub. Date: Jun. 15, 2006

(54) Biomarkers for intro-amniotic inflammation

(75) Inventors: Irina A Buhimschi, Troy, MI (US); Robert Christner, Toledo, OH (US)

Correspondence Address: FOLEY AND LARDNER LLP;SUITE 500, 3000 K STREET NW, WASHINGTON, DC, 20007, US

(73) Assignee: Ciphergen Biosystems, Inc.

(21) Appl. No.: 10/534,694

(22) PCT Filed: Nov. 13, 2003
(86) PCT No.: PCT/US03/36120

§ 371 Date: Jan. 17, 2006

Related U.S. Application Data
(60) Provisional application No. 60/426,096, filed on Nov. 14, 2002.

(51) Int. Cl. G01N 33/537; G01N 33/00

(52) U.S. Cl. 435/7.92; 436/86

(57) ABSTRACT

Biomarkers have been discovered that are capable of identifying intra-amniotic inflammation. A single biomarker or combination of biomarkers can be used to qualify the risk of preterm delivery in a patient, provided that at least one of the biomarkers is a calgranulin, preferably calgranulin A or C. The result is a rapid and reliable proteotmic approach to identifying intra-amniotic inflammation. In particular, the concentrations of the biomarkers correlate with the magnitude of intra-amniotic inflammation and, hence, of preterm delivery.

### BACKGROUND OF THE INVENTION

Premature birth is the leading cause of perinatal morbidity and mortality. Every year approximately 4.5 million premature babies are born worldwide, and, despite considerable advances in neonatal care, their mortality rate remains high. Moreover, survivors are at risk for long-term handicap, including developmental delay cerebral palsy, blindness deafness, and chronic lung disease. The societal burden of prematurity is underscored by the fact that, in the USA, the average cost per survivor with a birth weight of 900 grams or less (approximately 27 weeks) will exceed their medical and other supportive care expenses. Thus, the prevention of prematurity is the most important challenge to obstetrics and perinatal medicine. Its limited success has been attributed, in part, to the fact that premature parturition is a syndrome caused by multiple pathological processes such as infection, vascular disease, uterine over-distension, and chronic stress.

Intrauterine infection has emerged as a common and important cause of preterm delivery, as at least a third of all preterm births occur to mothers with microbial invasion of the amniotic cavity. Intrauterine infection often results in fetal infection with the development of the fetal inflammatory response syndrome, a risk factor for the impending onset of labor, short-term neonatal complications, and long-term handicaps, such as cerebral palsy and chronic lung disease.

Despite the compelling evidence that infection is causally linked to preterm delivery, antibiotic treatment of patients with premature labor has not proven effective in preventing preterm delivery or neonatal morbidity in most trials. A potential explanation is that many patients presenting with preterm labor do not have intrauterine infection and, hence, may not benefit from antimicrobial therapy. Moreover, intra-amniotic and fetal inflammation per se are linked to adverse outcome, even in the absence of microbiologically proven infection. Thus, the accurate and rapid identification of the mother with subclinical intrauterine inflammation is an urgent priority for the development of rational therapy.

Current evidence indicates that analysis of amniotic-fluid, which is normally sterile, is the most accurate means of determining the presence or absence of infection and/or inflammation. Available tests have limited sensitivity and specificity, however, and the results of standard microbiologic techniques, such as microbial culture, take time and are not available for immediate management decisions.

### SUMMARY OF THE INVENTION

To address these and other needs, the present invention provides a diagnostic assay and kit for detecting the presence of at least one biomarker indicative of intra-amniotic inflammation in a sample of amniotic fluid, comprising (A) mixing an adsorbent that binds at least one biomarker associated with intra-amniotic inflammation with a sample of amniotic fluid and then (B) monitoring the mixture for binding between said biomarker and the adsorbent, wherein the assay or kit detects at least one biomarker that is a calgranulin, particularly calgranulin A or calgranulin C. In one embodiment, the adsorbent is an antibody Immobilized on a solid substrate. The assay or kit using an antibody may be an ELISA in which an enzyme-antibody conjugate used to detect biomarker immobilized on the solid substrate. In some embodiments, the adsorbent is immobilized on a probe and the biomarker is detected by laser desorption/ionization mass spectrometry. In these embodiments, the adsorbent preferably is a hydrophobic adsorbent, more particularly a Ciphergen H4 probe or H50 probe. In preferred embodiments, the assays and kits 8 additionally tests for the presence of at least one defensin in said sample of amniotic fluid. In particular, the defensin may be HNP-1 alpha-defensin 1 or HNP-2 (alpha-defensin 2).

The invention provides a method for qualifying the risk of preterm delivery in a pregnant patient, comprised of analyzing a sample of amniotic fluid from the patient for a level of at least one calgranulin. Preferably, the method additionally comprises anaylzing the sample for the level of at least one defensin. In preferred embodiments, the calgranulin is calgranulin A or calgranulin C and the defensin is HNP-1 (alpha-defensin 1) or HNP-2 (alpha-defensin 2).

The invention further provides a method for qualifying the risk of preterm delivery in a pregnant patient, comprising (A) providing a spectrum generated by subjecting a sample of amniotic fluid from the patient to mass spectroscopic analysis that includes profiling on a biologically- or chemically-derivatized affinity surface, and (B) putting the spectrum through pattern-recognition analysis that is keyed to at least one peak indicative of the presence of a calgranulin in the sample. Preferably the pattern-recognition analysis additionally is keyed to at least one peak indicative of a defensin. In a preferred embodiment, the pattern-recognition analysis is keyed to at least one of calgranulin A or calgranulin C and at least one of HNP-1 (alpha-defensin 1) or HNP-2 (alpha-defensin 2). The preferred affinity surface is a Ciphergen H4 probe or H50 probe. The method is particularly useful in identifying the risk of preterm delivery in patients which do not have a white blood cell count that is elevated out of the normal range.

### BRIEF DESCRIPTION OF THE DRAWINGS

FIG. 1 is a flow chart of the distribution of patients used for “learning” Surface Enhanced Laser Desorption and Ionization (SELDI) profiles in amniotic fluid.

FIG. 2 presents representative protein mass-spectral profiles of “diseased,” “non-diseased,” and T-CRL patients who were studied during the learning phase. The discriminatory peaks composing the M (P1-P 13) and MR score (circled peaks) are shown within the three molecular weight areas of interest: 3300-3500 dalton (Da) under the CHCA-LL experimental protocol (a); 3500-3800 Da under CHCA-HL protocol (b); and 10-14 kDa under the SPA protocol (c).

FIG. 3 depicts patient data as follows: (a) Scattergrams represent the relationship between MR score of the amniotic fluid samples obtained from preterm patients (n=77) and the state of Intra-amniotic inflammation (WBC>100 cells/mm³) or intra-amniotic infection (positive amniotic fluid culture result). Closed circles represent the “diseased” patients. Open diamonds denote the “non-diseased” group. The rest of the patients are shown with open circles. (b) Survival analysis of the percent of undelivered patients after amniocentesis was performed In the preterm group of patients. Closed squares represent patients with MR scores of 3 or 4. Open squares are patients with MR scores of 0-2.

FIG. 4 presents data that identify peaks P1, P2, and P3 as neutrophil defensins (HNP-1-3) (a) and peaks P7 and P8 as calgranulins (b), based by on-chip antibody capture assays. Antibody-specific peaks are distinguished at the same mass with profiling tracings (H4), on spots where the antibody has been pre-adsorbed (Ab) but not on the spots pre-treated with IgG. The amniotic fluid samples were from representative “diseased” and “nondiseased” patients. Samples also were loaded onto Tricine gels and either stained with Coomasie blue lb) or processed for Western blotting, using the same antibody as was used for antibody capture: anti-HNP (insert at a) or Mac 387(c).

FIG. 5 presents a quantitative analysis of the peaks composing the MR score (log of normalized peak intensity) in the cohort of preterm patients (n=77), grouped by the presence or absence of intra-amniotic inflammation (+WBC: WBC>100/mm³) or microbiologically proven infection (+AFC: positive amniotic fluid culture results). The lines represent the means of the groups.

FIG. 6 depicts a quantitative analysis of a mixture of equal amounts of recombinant HNP-1 and HNP-2 on H4 spots. (a) SELDI profiles obtained after application of 1 μg (above) or 2 ng (below) of HNP 1-2 mixture Us) Log normalized peak intensity of the SELDI tracings versus the amount of HNP-1-2 mixture spotted. Each point represents the mean and SD from three individual spots. (c) Amniotic fluid HNP-1-3 concentration as measured by ELISA in the cohort of preterm patients (n=77) grouped by the presence or absence of intra-amniotic inflammation (+WBC: WBC>100/mm³) or microbiologically proven infection (+AFC: positive amniotic fluid culture results). The lines represent the means of the groups. (d) Correlation between the HNP-1-3 amount in amniotic fluid measured by ELISA (log) and log normalized intensity of the P2 peak (HNP-1) by SELDI.

### DETAILED DESCRIPTION OF THE PREFERRED EMBODIMENTS

Biomarkers have been discovered, each associated with intra-amniotic inflammation. In the present context, a “biomarker” is an organic biomolecule, particularly a polypeptide or protein, which is differentially present in a sample taken from a subject having intra-amniotic inflammation as compared to a comparable sample taken from a “normal” subject that does not have intra-amniotic inflammation. A biomarker is differentially present in samples from a normal patient and a patient having intra-amniotic inflammation, respectively, if it is present at an elevated level or a decreased level in latter samples as compared to samples of normal patients.

The biomarkers of the invention are capable of identifying intra-amniotic inflammation. A single biomarker or combination of biomarkers (“biomarker profile”) can be employed, in accordance with the invention, provided that at least one of the biomarkers is a calgranulin, preferably calgranulin A or C. The biomarkers and biomarker profiles of the invention can be used to qualify the risk of preterm delivery in a patient.

Thus, the present invention provides a rapid and reliable proteomic approach to identifying intra-amniotic inflammation which can lead to preterm delivery. This is the first proteomic characterization of amniotic fluid in premature labor, and detailed analyses of the biomarkers, permits characterization and quantitative validation of the changes involved. In particular, the concentrations of the biomarkers correlate with the magnitude of the biological phenomena of interest, namely, Intra-amniotic inflammation and preterm delivery.

During the second trimester of pregnancy fetal urine and lung liquid are the two major inflows into the anmiotic cavity. In pregnancies complicated with fetal inflammation, moreover, the amniotic fluid contains neutrophils, which are for the most part of fetal and not of maternal origin, and the concentrations of inflammatory mediators in this fluid predict the likelihood of impending preterm delivery and adverse neonatal outcome better than maternal blood.

The biomarkers of the present invention are primarily defensins and calgranulins. Defensins are proteins of the innate immune system. The three principle human neutrophil defensins, HNP 1-3, belong to the family of unique to neutrophils and account for 99 percent of the defensin content in these cells. HNP-1, -2 and -3 belong to the family of cationic, trifsulfide-containing microbicidal peptides. Their production and release is induced by cytokines and microbial products such as lipopolysaccharide, a component of the cell wall of Gram negative bacteria.

Calgranulins are members of the S100 group of proteins, which are calcium-binding proteins that contain two canonical EF-hand structural motifs. They have received increasing attention due to their possible involvement In diseases such as Alzheimer's, cancer, cardiomyopathy, psoriasis, rheumatoid arthritis, and other inflammatory disorders. S100 A8 (calgranulin A) and S100 A9 (calgranulin B) can combine to form homodimers and heterodimers, which also have antimicrobial properties. The approach developed by the inventors, described in greater detail below, has allowed for the identification of biomarkers, such as calgranulin C, which has not been previously studied in preterm parturition or intra-amniotic infection.

The present invention comprehends the proteomic analysis of amniotic fluid, to obtain semi-quantitative information that correlates with the magnitude of the inflammatory phenomenon, as determined by the intensity of intrauterine inflammation (i.e., correlation with white blood cell counts) and the clinical outcome (i.e., relationship between duration of pregnancy and MR score, described below). In, particular, the present invention encompasses a means for predicting preterm delivery, based on an analysis of patterns of particular-defensins and calgranulins. A biomarker profile identified in accordance with this invention reliably indicates the presence or absence of inflammation, which is of major importance because, as noted above, intra-amniotic inflammation is a risk factor for preterm delivery, short-term complications of prematurity, and long-term squealae such as cerebral palsy and chronic lung disease.

In a preferred embodiment of the invention, moreover, such proteomic analysis is combined with molecular microbiological techniques to detect microorganisms that are responsible for detected inflammation, thereby to inform selection of an antimicrobial therapy. That is, a proteomic analysis according to the invention can identify a patient as suffering intra-amniotic inflammation, and samples of amniotic fluid from the patient determined can be tested, in conventional manner, to identify pathogenic microorganisms responsible to the inflammation. Thus, the test data can help determine an antibiotic regimen that is likely to be effective against the identified microorganisms.

Proteomic analysis of amniotic fluid, In accordance with the invention, provides a rapid, simple and reliable means of Identifying the patient in premature labor with intra-amniotic inflammation, who are at risk for impending preterm delivery. Thus identified, this cohort of patients may be selected to test specific interventions to eradicate infection and/or to modulate the inflammatory response associated with adverse outcome.

The biomarkers according to the present invention were identified by comparing mass spectra of samples derived from amniotic fluid from two groups of pregnant subjects, subjects with intra-amniotic inflammation and normal subjects. The subjects were diagnosed according to standard clinical criteria.

These two pools were used in a wide range of dilutions to test various chip surfaces, produced by Ciphergen Biosystems (Fremont, Calif.), for optimal discriminatory performance, including reverse phase H4, a hydrophobic surface with C-16 long chain aliphatic residues; SAX 2, a strong anion exchanger; WCX2, a quaternary ammonium, weak cation exchanger; IMAC, carboxylate residues; metal affinity). For H4 chip surfaces, optimization involved additional hydrophobic washes of acetonitrile gradients (10% to 75%). A procedure where 2 μl of amniotic fluid, diluted 10-fold in phosphate buffer saline (PBS), was placed on a spot of a 24-spot H4 array and incubated in a humidified box, avoiding desiccation, was found to be optimal for individual peak detection and low signal-to-noise (S/N) ratios. As described In greater detail below, however, other chips can be used, so long as they have binding characteristics that are suitable for binding the biomarkers of the invention.

According to one aspect of the invention, therefore, the detection of biomarkers for diagnosis of intra-amniotic inflammation entails contacting a sample of amniotic fluid from a patient with a substrate, having an adsorbent thereon, under conditions that allow binding between the biomarker and the adsorbent, and then detecting the biomarker bound to the adsorbent by gas phase ion spectrometry, for example, mass spectrometry. Other detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry). Immunoassays in various formats, such as ELISA, likewise can be adapted for detection of biomarkers captured on a solid phase, in accordance with the present invention (see below).

A preferred mass spectrometric technique for use in the invention is Surface Enhanced Laser Desorption and Ionization (SELDI), as described, for example, in U.S. Pat. No. 5,719,060 and No. 6,225,047, both to Hutchens and Yip, in which the surface of a probe that presents the analyte (here, one or more of the biomarkers) to the energy source plays an active role in desorption ionization of analyte molecules. In this context, “probe” refers to a device adapted to engage a probe interface and to present an analyte to ionizing energy for ionization and introduction into a gas phase, ion spectrometer, such as a mass spectrometer. A probe typically includes a solid substrate, either flexible or rigid, that has a sample-presenting surface, on which an analyte is presented to the source of ionizing energy.

One version of SELDI, called Surface-Enhanced Affinity Capture” or “SEAC,” involves the use of probes comprised of a chemically selective surface (“SELDI probe”). A “chemically selective surface” is one to which is bound either the adsorbent, also called a “binding moiety” or “capture reagent,” or a reactive moiety that is capable of binding a capture reagent, e.g., through a reaction forming a covalent or coordinate covalent bond.

The phrase “reactive moiety” here denotes a chemical moiety that is capable of binding a capture reagent. Epoxide and carbodiimidizole are useful reactive moieties to covalently bind polypeptide capture reagents such as antibodies or cellular receptors. Nitriloacetic acid and iminodiacetic acid are useful reactive moieties that function as chelating agents to bind metal ions that interact non-covalently with histidine containing peptides. A “reactive surface” is a surface to which a reactive moiety is bound. An “adsorbent” or “capture reagent” can be any material capable of binding a biomarker of the invention. Suitable adsorbents for use in SELDI, according to the invention, are described in U.S. Pat. No. 6,225,047, supra.

One type of adsorbent is a “chromatographic adsorbent,” which is a material typically used in chromatography. Chromatographic adsorbents include, for example, ion exchange materials, metal chelators, immobilized metal chelates, hydrophobic interaction adsorbents, hydrophilic interaction adsorbents, dyes, mixed mode adsorbents (e.g., hydrophobic attraction/electrostatic repulsion adsorbents). “Biospecific adsorbent” is another category, for adsorbents that contain a biomolecule, e.g., a nucleotide, a nucleic acid molecule, an amino acid, a polypeptide, a simple sugar, a polysaccharide, a fatty acid, a lipid, a steroid or a conjugate of these (e.g., a glycoprotein, a lipoprotein, a glycolipid). In certain instances the biospecific adsorbent can be a macromolecular structure such as a multiprotein complex, a biological membrane or a virus. Illustrative biospecific adsorbents are antibodies, receptor proteins, and nucleic acids. A biospecific adsorbent typically has higher specificity for a target analyte than a chromatographic adsorbent.

Another version of SELDI is Surface-Enhanced Neat Desorption (SEND), which involves the use of probes comprising energy absorbing molecules that are chemically bound to the probe surface (“SEND probe”). The phrase “Energy absorbing molecules” (EAM) denotes molecules that are capable of absorbing energy from a laser desorption Ionization source and, thereafter, contributing to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyaceto-phenone derivatives. The category also includes EAMs used in SELDI, as enumerated, for example, by U.S. Pat. No. 5,719,060 and U.S. 60/351,971, filed Jan. 25, 2002.

Another version of SELDI, called Surface-Enhanced Photolabile Attachment and Release (SEPAR), involves the use of probes having moieties attached to the surface that can covalently bind an analyte, and then release the analyte through breaking a photolabile bond in the moiety after exposure to light, e.g., to laser light. For instance, see U.S. Pat. No. 5,719,060. SEPAR and other forms of SELDI are readily adapted to detecting a biomarker or biomarker profile, pursuant to the present invention.

The detection of the biomarkers according to the invention can be enhanced by using certain selectivity conditions, e.g., adsorbents or washing solutions. The phrase “wash solution” refers to an agent, typically a solution, which is used to affect or modify adsorption of an analyte to an adsorbent surface and/or to remove unbound materials from the surface. The elution characteristics of a wash solution can depend, for example, on pH, ionic strength, hydrophobicity, degree of chaotropism, detergent strength, and temperature.

Pursuant to one aspect of the present invention, a sample is analyzed by means of a “biochip,” a term that denotes a solid substrate, having a generally planar surface, to which a capture reagent (adsorbent) is attached. Frequently, the surface of a biochip comprises a plurality of addressable locations, each of which has the capture reagent bound there. A biochip can be adapted to engage a probe interface and, hence, function as a probe, which can be inserted into a gas phase ion spectrometer, preferably a mass spectrometer. Alternatively, a biochip of the invention can be mounted onto another substrate to form a probe that can be inserted into the spectrometer.

A variety of biochips is available for the capture of biomarkers, in accordance with the present invention, from commercial sources such as Ciphergen Biosystems (Fremont, Calif.), Packard BioScience Company (Meriden Conn.), Zyomyx (Hayward, Calif.), and Phylos (Lexington, Mass.). Exemplary of these biochips are those described in U.S. Pat. No. 6,225,047, supra, and No. 6,329,209 (Wagner et al.), and in PCT publications WO 99/51773 (Kuimelis and Wagner) and WO 00/56934 (Englert et al.).

More specifically, biochips produced by Ciphergen Biosystems have surfaces, presented on an aluminum substrate in strip form, to which are attached, at addressable locations, chromatographic or biospecific adsorbents. The surface of the strip is coated with silicon dioxide.

Illustrative of Ciphergen ProteinChip® arrays are biochips H4, SAX-2, WCX-2, and IMAC-3, which include a functionalized, cross-linked polymer in the form of a hydrogel, physically attached to the surface of the biochip or covalently attached through a silane to the surface of the biochip. The H4 biochip has isopropyl functionalities for hydrophobic binding. The SAX-2 biochip has quaternary ammonium functionalities for anion exchange. The WCX-2 biochip has carboxylate functionalities for cation exchange. The IMAC-3 biochip has nitriloacetic acid functionalities that adsorb transition metal ions, such as Cu⁺⁺ and Ni⁺⁺, by chelation. These immobilized metal ions, in turn, allow for adsorption of biomarkers by coordinate bonding.

In keeping with the above-described principles, a substrate with an adsorbent is contacted with the sample, containing amniotic fluid, for a period of time sufficient to allow biomarker that may be present to bind to the adsorbent. After the incubation period, the substrate is washed to remove unbound material. Any suitable washing solutions can be used; preferably, aqueous solutions are employed.

An energy absorbing molecule then is applied to the substrate with the bound biomarkers. As noted, an energy absorbing molecule is a molecule that absorbs energy from an energy source in a gas phase ion spectrometer, thereby assisting in desorption of biomarkers from the substrate. Exemplary energy absorbing molecules include, as noted above, cinnamic acid derivatives, sinapinic acid and dihydroxybenzoic acid. Preferably sinapinic acid is used.

The biomarkers bound to the substrates are detected in a gas phase ion spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Data generated by desorption and detection of markers can be analyzed with the use of a programmable digital computer. The computer program analyzes the data to indicate the number of markers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set as zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Software used to analyze the data can include code that applies an algorithm to the analysis of the signal to determine whether the signal represents a peak in a signal that corresponds to a biomarker according to the present Invention. The software also can subject the data regarding observed biomarker peaks to classification tree or ANN analysis, to determine whether a biomarker peak or combination of biomarker peaks is present that indicates a diagnosis of Intra-amniotic inflammation.

In another aspect, the present invention provides kits for aiding in the diagnosis of intra-amniotic inflammation, which kits are used to detect biomarkers according to the invention. The kits screen for the presence of biomarkers and combinations of biomarkers that are differentially present in samples from subjects with intra-amniotic inflammation.

In one embodiment, the kit comprises a substrate having an adsorbent thereon, wherein the adsorbent is suitable for binding a biomarker according to the invention, and a washing solution or instructions for making a washing solution, in which the combination of the adsorbent and the washing solution allows detection of the biomarker using gas phase ion spectrometry. In preferred embodiments, the kit comprises a immobilized metal affinity capture chip, such as the H4 chip.

In another embodiment, a kit of the invention may include a first substrate, comprising an adsorbent thereon, and a second substrate onto which the first substrate is positioned to form a probe, which can be inserted into a gas phase ion spectrometer. In another embodiment, an inventive kit may comprise a single substrate that can be inserted into the spectrometer.

In a further embodiment, such a kit can comprise instructions for suitable operational parameters in the form of a label or separate insert. For example, the instructions may inform a consumer how to collect the sample or how to wash the probe.

The biomarkers according to the invention also are useful in the production of other diagnostic assays for detecting the presence of the biomarker In a sample. For example, such assays may comprise, as the “adsorbent,” “binding moiety,” or “capture reagent” an antibody to one or more of the biomarkers, with the proviso that at least one of the biomarkers is a calgranulin. The antibody is mixed with a sample suspected of containing the biomarkers and monitored for biomarker-antibody binding. The biomarker antibody Is labelled with a radioactive or enzyme label. In a preferred embodiment, the biomarker antibody is immobilized on a solid matrix such that the biomarker antibody is accessible to biomarker in the sample. The sample then is brought into contact with the surface of the matrix, and the surface is monitored for biomarker-antibody binding.

For example, the biomarker can be detected in an enzyme-linked Immunosorbent assay (ELISA), in which biomarker antibody is bound to a solid phase and an enzyme-antibody conjugate is used to detect and/or quantify biomarker present in a sample. Alternatively, a western blot assay can be used in which solubilized and separated-biomarker is bound to nitrocellulose paper. The combination of a highly specific, stable liquid conjugate with a sensitive chromogenic substrate allows rapid and accurate identification of samples. All solutions required for blocking and washing the membrane and for diluting antibodies are provided. The biomarker is detected by an enzyme or label-conjugated anti-immunoglobulin (Ig), such as horseradish peroxidase-Ig conjugate by incubating the filter paper in the presence of a precipitable or detectable substrate. Western blot assays have the advantage of not requiring purity greater than 50% for the desired biomarker(s). Descriptions of ELISA and western blot techniques are found in Chapters 10 and 11 of Ausubel et al. (eds.), CURRENT PROTOCOLS IN MOLECULAR BIOLOGY (John Wiley and Sons, 1988).

The present invention is further described by reference to the following, illustrative examples.

Study Population

A total of 114 amniotic fluid samples from distinct patients were analyzed in this study. Ninety samples were used to establish proteomic patterns with diagnostic value and 24 samples were used to test and validate the algorithm. Amniotic fluid was obtained by amniocentesis performed for the assessment of the microbiological status of amniotic cavity and/or fetal lung maturity. Samples from patients at term were obtained at the time of elective caesarian section. Preterm labor was defined as the presence of uterine contractions (at least 3 in 10 min.) or advanced cervical dilatation at less than 37 weeks of gestation. Preterm premature rupture of membranes (PROM) was diagnosed by sterile speculum examination confirming leakage of amniotic fluid in the vagina, and positive ferning and nitrazine test results. Samples for research were collected under IRB protocols approved by Wayne State University after written informed consent was obtained. The utilization of these samples for research was approved by the IRB of the National Institute of Child Health and Human Development. Cultures for aerobic, anaerobic bacteria and genital Mycoplasmas, Gram stain, counts of white and red blood cells were performed immediately after collection. The remaining amniotic fluid was centrifuged at 700 g, 4° C. for 10 min. and then was stored in aliquots at −80° C. until analysis.

Protein Profiling Protocol for SELDI-TOF Mass Spectrometry

Preliminary experiments were conducted to optimize the protein profiling protocol. Two pools of amniotic fluid were generated, using samples from two extreme “diseased” and “non-diseased” groups, as shown in Table I, below.

These two pools were used In a wide range of dilutions to test various chip surfaces for optimal discriminatory performance (reverse phase H4: hydrophobic surface with C-16 long chain aliphatic residues; strong anion exchanger SAX2: quaternary ammonium; weak cation exchanger WCX2: carboxylate residues; metal affinity: IMAC). For H4 chip surfaces, optimization involved additional hydrophobic washes of acetonitrile gradients (10% to 75%). We learned that a procedure where 2 μl of amniotic fluid diluted 10-fold in phosphate buffer saline (PBS) placed on a spot of a 24-spot H4 array, a product of Ciphergen Biosystems (Fremont, Calif.), and incubated in a humidified box to avoid desiccation was optimal for individual peak detection and low signal-to-noise (S/N) ratios. After one hour, the sample was aspirated and the spots washed individually with 3 volumes of 5 μl 25% aqueous acetonitrile solution, left to air dry and then overlaid with matrix solution (energy absorbing molecule) diluted in 0.5% trifluoroacetic acid/50% acetonitrile.

TABLE I
Patient chart data and amniotic fluid analysis of preterm patients used for “learning” SELDI profiles

| | PT-CRL | +AFC + WBC | | | |
|---|---|---|---|---|---|
| | “non-diseased” | “diseased” | −AFC + WBC | +AFC − WBC | −AFC − WBC |
| | n = 17 | n = 21 | n = 7 | n = 8 | n = 24 |
| Patient characteristics | | | | | |
| Age (years: mean ± SD) | 27 ± 7 | 27 ± 7 | 24 ± 5 | 28 ± 4 | 24 ± 5 |
| Ethnicity (% African-American) | 82% | 85.7% | 85.7% | 100% | 96% |
| Parity (median [range]) | 1 [0-2] | 1 [0-7] | 1 [0-6] | 2 [0-6] | 1 [0-8] |
| Gravidity (median [range]) | 4 [3-10] | 3 [1-10] | 5 [1-7] | 5.5 [1-8] | 2 [1-11] |
| Patients with history of preterm | 3/12 | 3/11 | 3/5 | 3/7 | 6/13 |
| delivery/multiparours patients | | | | | |
| Admission-Amniocentesis-Delivery | | | | | |
| GA at amniocentesis (wks ± SD) | 29.7 ± 3 | 27.5 ± 4 | 26.5 ± 2 | 32.1 ± 1 | 30.0 ± 3 |
| GA at delivery (wks ± SD) | 38.5 ± 1 | 27.8 ± 4 | 27.7 ± 3 | 33.0 ± 1 | 31.8 ± 4 |
| PPROM (n [%]) | 0 | 8 [38%] | 4 [57%] | 7 [87.5%] | 8 [33%] |
| Induced/spontaneous labour | 2/15 | 8/11 | 0/6 | 5/3 | 8/16 |
| Cesarean deliveries | 1 | 4 | 1 | 0 | 2 |
| Birth weight (grams ± SD) | 3290 ± 491 | 1117 ± 589 | 1073 ± 395 | 1952 ± 302 | 1858 ± 515 |
| Amniotic fluid characteristics | | | | | |
| WBC (cells/mm³: median [range]) | 10 [0-80] | 1800 [335-19200] | 520 [200-14800] | 23 [3-90] | 3 [0-62] |
| Positive Gram stain | 0 | 13 | 0 | 3 | 1 |
| Histologic chorioamnionitis and/or funisitis | 1 | 19 | 7 | 3 | 5 |

GA: gestational age;
WBC: white blood cell count (+WBC: WBC count >100 cells/mm3);
AFC: Amniotic fluid cultures; (+AFC: positive amniotic fluid culture results).
The “diseased” and “non-diseased” groups are the two extreme groups used for learning SELDI profiles.

Diluted amniotic fluid from each patient was assigned to duplicate chips and on each chip two spots were covered with 2 μl of PBS alone. The matrix consisted of either 1 μl of a 20% saturated solution of α-cyano-4-hydroxycinnamic acid (CHCA), on one set of chips or two sequential applications of 0.5 μl saturated solution of sinnapinic acid (SPA) on the other. The chips were allowed to air-dry and then were read in a Protein Biology System® II (PBS II) SELDI-TOF mass spectrometer (Ciphergen Biosystems), using the ProteinChip® software, versions 2.1 b and 3. The chip covered with CHCA was analyzed first using a low-laser intensity spot protocol (CHCA-LL: laser intensity 220, mass ranging from 0 to 20,000 Da, optimized between 1000 and 10,000 with detector sensitivity 6, mass focus 3300 Da, 20 shots fired and averaged for every 5-th position from starting from position 20 to 80), followed by a high-laser intensity spot protocol (CHCA-HL: laser intensity 240, detector sensitivity 10, 25 shots fired and averaged for every 5-th position from position 22 to 82). The chip covered with SPA was analyzed with a single spot protocol (SPA: laser intensity 285, mass ranging from 0 to 200,000 Da, optimized between 20,000-90,000 Da with mass focus at 26,500 Da, detector sensitivity 10, and 20 shots fired and averaged for every 5-th position from position 20 to 80). The PBS II instrument was calibrated externally against four molecular weight peptide standards: arg-8-vasopressin, bovine insulin 13 chain, human insulin, and hirudin.

Procedures for Biomarker Identification and Quantification

Isoelectric points (pH where the protein has no net charge) for the peaks of interest were estimated by spotting diluted amniotic fluid onto WCX2 and SAX2 chips and washing individual spots with solutions of incremental pHs (50 mM sodium acetate pH=4, 50 mM sodium phosphate pH=6, 50 mM Tris pH=8 or 50 mM sodium carbonate pH=10) before SELDI analysis. If the pH of the buffer solution is below its isoelectric point, a protein will bear a net positive charge binding preferentially to the WCX2. If the pH of the buffer solution is above its isoelectric point, it will bind preferentially to the SAX2 surface. Thus, the isoelectric point of a protein of interest would be between the pH of the buffer solutions where a peak appears on the SAX2 and the WCX2 chip, respectively.

In-gel trypsin digest. ID-SDS-PAGE electrophoresis was carried out on precast 10-20% tris-tricine gels (5-well gels, InVitrogen, Carlsbad, Calif.). Samples were prepared by boiling amniotic fluid for 5 mm. with an equal volume of Tricine loading buffer (BioRad, LaJolla, Calif.) under non-reducing conditions. The gels were loaded with 20 μl sample volume per well, run at 120V, stained with 0.1% Comassie Blue R250 in 40% methanol/10% acetic acid for 1 hour and then destained with repeated changes of 40% methanol/10% acetic acid. Low molecular weight markers (Ultralow Color marker, product of Sigma, St Louis, Mo.) or a mixture of mass spectrometry molecular weight standards (0.5 nmols of bovine cytochrome C and 0.5 nmols of bovine ubiquitin, Ciphergen Biosystems) were loaded on gels along with the amniotic fluid samples.

Bands of interest were precisely cut out, using a scalpel blade, and were minced, and in-gel tryptic digestion then was performed, using a peptide mapping kit, product of Ciphergen Biosystems (Fremount, Calif.). At the end of the procedure 50 μl of proteomics sequencing grade trypsin (Sigma) containing 0.2 μg enzyme in 0.4 mM HC1/25 mM ammonium bicarbonate was added to each tube containing the gel pieces and incubated for 16 hours in a 37° C. oven. A piece of blank gel was processed along with the protein bands to differentiate between products resulting from trypsin autolysis and fragments from the protein of interest. Peptide maps were read manually in the PBS II Instrument after 1.5° l of each digest was dried directly onto H4 spots and then overlaid with 1 μl 10% saturated CHCA solution.

Peptide elution. A few gel pieces from each band were placed in a sonic bath in 20 fit of 50% formic acid/25% acetonitrile/15% isopropanol for 30 minutes. The solution then was removed and evaporated under vacuum, and the eluted proteins resuspended in 10 μl water, of which 5 μl were dried down onto H4 spots, overlaid with two applications of 0.5 μl saturated SPA solution and read manually in the PBS II instrument.

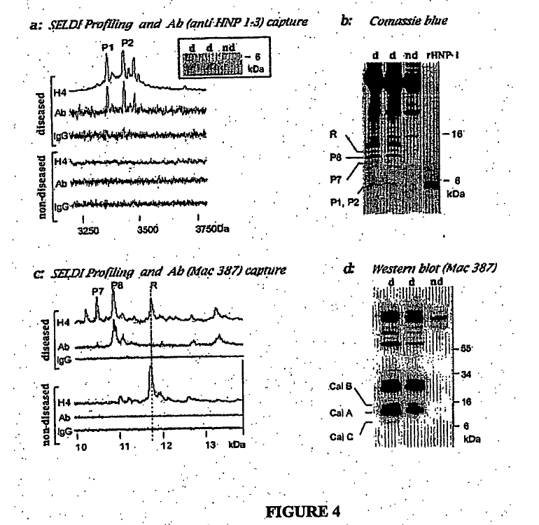

Gel staining and western blotting. 5 μl of amniotic fluid diluted 1:1 with tricine sample buffer (Bio-Rad) were boiled for 5 min. and loaded under onto 16% continuous (for HNP-1-3; see FIG. 4b) or 10-20% gradient (for calgranulins; see FIG. 4c) precast tricine, product of InVitrogen Corporation (Carlsbad, Calif.). Gels either were stained with Comassie blue (FIG. 4b) or were transferred electrophoretically to PVDF membrane filters for western blotting (FIG. 4c). Briefly, filters were blocked with 5% milk and then incubated with either mouse Mac 387 monoclonal antibody for calgranulins (1:1000 dilution; Labvision, Fremont, Calif.) or rabbit polyclonal anti HNP-1-3 (1:1000 dilution, rabbit anti-human HNP-1-3; Abcam, Cambridge, UK) for 1 h at 25° C. Detection was performed using appropriate horseradish peroxidase-linked secondary antibody and ECL-kit (Amersham Biosystems).

On-chip immunoassay. Spots on PS20 arrays (epoxy-activated for covalent immobilization of proteins) where incubated for 1 h with 2, affinity purified antibody solution (Mac 387 for calgranulin capture or anti human HNP-1-3) at 1 mg/ml diluted in PBS as purchased from the manufacturer. Paired chips where spotted with mouse or rabbit IgG, as appropriate, in a similar concentration. Unreacted active sites were blocked by incubation with 4 μl per spot 1 M Tris, pH=9 for 20 min. at room temperature. The chips were then washed with PBS supplemented with up to 850 mM NaCl and 0.05% Tween-20 (binding buffer). A 5 μl sample (diluted progressively in binding buffer from 1:10 to 1:80 for calgranulin or 1:500 to 1:64,000 for HNP capture) was incubated on the pre-treated spots. After one hour, the spots were vigorously washed, first with binding buffer and then with 10 mM HEPES, were allowed to air-dry, were covered with appropriate matrix solution, and then were read in the PBS II system.

ELISA. Concentrations of HNP-1-3 in amniotic fluid were measured with a commercially available enzyme-linked immunosorbent assay (HyCult Biotechnologies, Uden, The Netherlands) with a sensitivity of 19.5 pg/mL. Intra-assay and inter-assay coefficients of variation were <2%.

Statistical Analysis

Data were tested for normality using the Kolmogorov-Smirnov test and compared with Mann-Whitney test (nonparametric), Student's t test (parametric), or one-way ANOVA (parametric for comparisons among multiple groups). Comparisons between proportions were performed using the Fisher's exact test. Receiver operating characteristic (ROC) curve analysis, inter-rate agreement and kappa calculations were performed using MedCalc statistical software (MedCalc, Broekstraat, Belgium).

Results

(1) “Learning” SELDI Protein Profiles in Amniotic Fluid

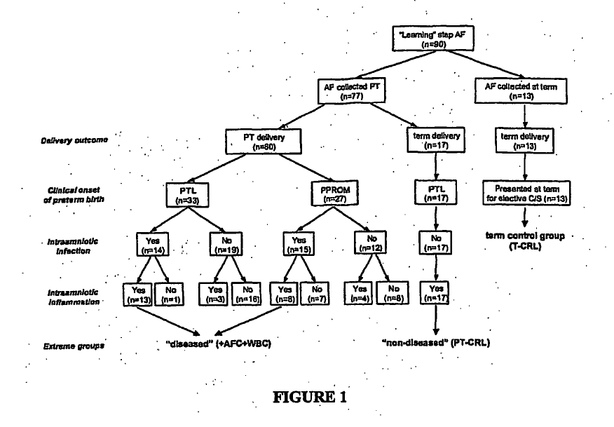

We first analyzed amniotic fluid samples from 90 pregnant women (FIG. 1). Seventy-seven were obtained from patients with the diagnosis of preterm labor (n=50) or preterm PROM (n=27); (average gestational age: 29 weeks [95% Cl: 28-30]) and 13 from patients at term, not in labour (average gestational age: 39 weeks [95% Cl: 39-40]). The first step was to generate two groups with extreme clinical and biological characteristics, namely, a “diseased” and a “non-diseased” group. Patients who delivered a preterm neonate and had evidence of infection and inflammation were selected to represent the “diseased” group. Infection was defined as a positive amniotic fluid culture for microorganisms whereas intra-amniotic inflammation was defined as an amniotic fluid WBC count >100 cells/mm³. The “non-diseased” group constituted of patients with premature labour with intact membranes who subsequently delivered a term neonate without complications (preterm control group: PT-CRL). Patients at term at the time of amniotic fluid retrieval represented an additional control group (term control group: T-CRL). The distribution of patients according to clinical presentation (preterm parturition or term gestation), preterm delivery (<37 weeks), amniotic fluid cultures and amniotic fluid WBC count is illustrated in FIG. 1. The clinical characteristics of the subgroups are displayed in Table I. The extreme “diseased” and “non-diseased” groups are in the first two columns.

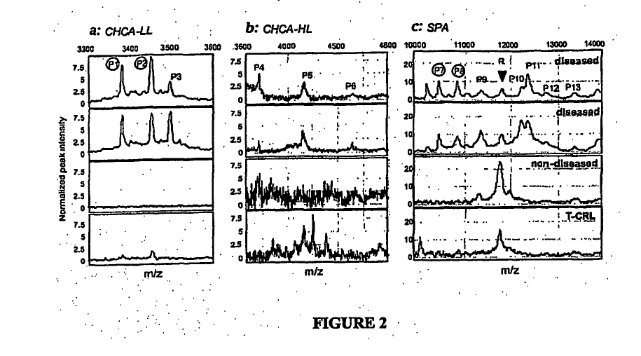

Preliminary data mining consisted of visual inspection of the SELDI protein profile tracings followed by evaluation with the “biomarker wizard tool” at a 0.3% mass accuracy and biomarker statistics. This analysis suggested that informative peaks were clustered within three m/z (ratio mass/charge) areas. These areas of interest were between 3300-3600 Da in the CHCA-LL spectra, 3600-5000 Da in the CHCA-HL spectra and 10,000-14,000 Da with SPA (FIG. 2). Conspicuous peaks in these zones were selected manually and the spectra from each patient verified for accuracy of peak identification. The m/z value, normalized intensity and signal to noise ratio (S/N) for the selected peaks were extracted. Next, we established a stepwise strategy to define rigorous filter preferences for our diagnostic proteomic profile. The criteria included the following: (1) all peaks should be present in the “diseased” state. Thus, the search was for new peaks in “diseased” patients, rather than disappearance or decrease of peaks normally present in “non-diseased” patients; (2) the peaks of the profile should have been detected on at least two different laser intensities or matrix protocols; (3) all peaks in the profile should be significantly different (in logarithm of normalized-intensity) at least at a level of p<0.0001 between the “diseased” and “non-diseased” groups; (4) only parent peaks were considered (singly ionized, least oxidized); (5) peaks should not have occurred in areas where the baseline noise in “non-diseased” individuals is significantly elevated; and (6) the final diagnostic profile should be parsimonious.

After applying the first four criteria, 13 candidate peaks with potential discriminatory value emerged (FIG. 2). To objectively score the peaks as present or absent, the evaluation of the S/N ratio was undertaken. The cut-off used for selection was the mean±2 standard deviations of the S/N ratio for each corresponding mass in the “non-diseased” group. Boolean indicators were then assigned: a value of 0 was used if a peak was absent or below the cut-off and a value of 1 was assigned for peaks above the cut-off. The sum of Boolean indicators was computed for each patient and is referred as the M score (Mass score). This score represents the sum of observations in the three different matrix protocols (for example, the first two patients in FIG. 2 have M scores of 12; while the third and the fourth have scores of 0 and 3, respectively).

To select the peaks not occurring in areas of baseline noise in “non-diseased” individuals (fifth criterion), we analyzed the noise level for each matrix protocol (C HCA-LL, CHCA-HL and SPA) at the 13 corresponding average masses on the spectra from the intermingled spots with PBS instead of amniotic fluid (n-20). We eliminated the peaks where the average noise level (S/N at the corresponding m/z value) in “non-diseased” group was significantly higher (p<0.05) than the noise level with PBS alone. This criterion was chosen to minimize overlap of informative peaks with those present nearby in the tracing of normal women. Another advantage of this approach was that objective quantitative and qualitative analysis of the peaks of interest was now possible and independent of the availability of “non-diseased” samples, which would be a substantial obstacle in the clinical implementation of the test.

After sequentially applying the filtering strategy outlined above, we obtained a set of four peaks, which defined the final proteomic profile. The sum of 0 or 1 values for each of these four peaks computed the MR score (Mass restricted score), ranging from 0 to 4 (the peaks selected for calculation of the MR are displayed in FIG. 2 within the circle; the first two patients have MR scores of 4, the third and the fourth of 0 and 2, respectively). Therefore, each patient had two scores: the M score and the MR score. In Table II, the peaks composing the M and the MR score are listed with their observed mass, as derived only from the peaks with a Boolean indicator of 1 (i.e., peak is present).

In addition to the 13 initial peaks composing the M proteomic profile, a distinct peak of approximately 11.7 kDa (defined as reference or R peak, FIG. 2) was present in all fluids. This peak proved useful for the quick visual orientation to the m/z axis of the SPA spectra.

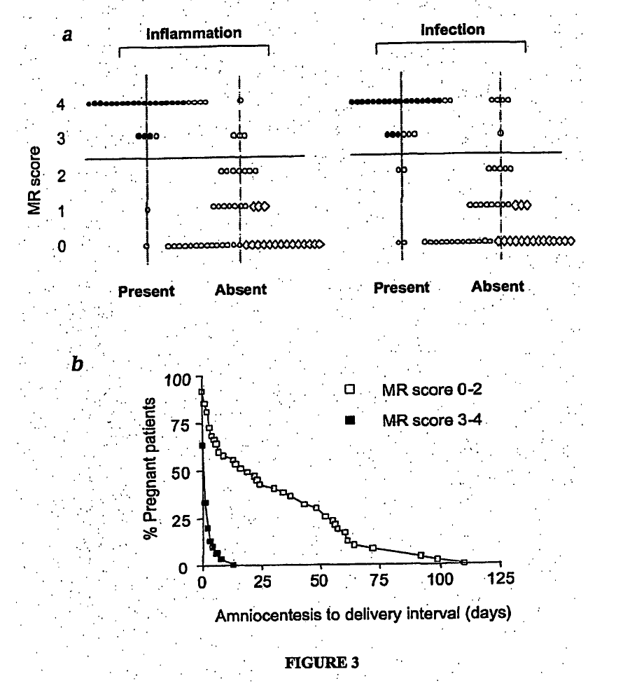

To test the diagnostic performance of the M and MR scores in the discrimination of “diseased” and “non-diseased” states, receiver operating characteristic curve analysis was performed. The M and MR scores performed identically in delineating between patients with infection and inflammation and those with premature labor who delivered at term. This indicates that the 4 peaks composing the MR score are sufficient for the stated purposes (i.e., parsimony). All patients with preterm labor who delivered at term had an MR score of either 0 or 1 (open diamonds in FIG. 3a). All patients with inflammation and infection (closed circles in FIG. 3a) had an MR score of 3 or 4. Thus, an MR score >2 had 100% sensitivity and 100% specificity in the discrimination of these two extreme clinical conditions.

To determine whether the MR score could discriminate between preterm patients with intra-amniotic inflammation and those with microbiologically proven infection, the samples from the entire population were evaluated. An MR score over 2 had a sensitivity of 92.9% (26/28) and a specificity of 91.8% (45/49) in the detection of intrauterine inflammation (area under the ROC curve 0.948, SE=0.031, 95% Cl: 0.871 to 0.985). Two patients had WBC>100/mm³ with low MR scores (false negative for inflammation) (FIG. 3a). Both had negative microbial cultures. One presented with preterm PROM at 28 weeks of gestation had 200 WBC/mm³ and delivered prematurely after 9 days. The other patient presented at 27 weeks of gestation had 375 WBC/mm³ and delivered at 30 weeks of gestation during a second admission for abdominal trauma. There were 4 women with WBC<100/mm³ who had MR scores of 3 or 4 (false positive for inflammation). All presented with preterm-PROM at 30-33 weeks of gestation, delivered within 3 days and in all Ureaplasma urealyticum or Mycoplasma hominis were isolated from the amniotic fluid.

An MR score greater than 2 identified intra-amniotic infection with 86.2% sensitivity (25/29), 89.6% specificity (43/48) (area under the ROC curve=0.893, SE=0.042, 95% Cl: 0.802 to 0.952) (FIG. 3a). Four patients had positive amniotic fluid cultures for microorganisms but did not display the characteristic profile (i.e., false negative for infection: 2 patients had a score of 0 and 2 an MR score of 2 due to P1 and P2 presence). The two patients with an MR of 0 had amniotic fluid WBC counts below 5 WBC/mm³ (no intra-amniotic inflammation). One patient was admitted at 33 weeks of gestation with preterm PROM and delivered within two days after labor induction. The amniotic fluid culture was positive for Streptococcus agalactiae, the neonate weighed 2160 grams, had Apgar scores of 9/9 and no complications. The second patient was admitted at 29 weeks of gestation with preterm labour and intact membranes, had a positive amniotic fluid culture for Streptococcus viridans, and delivered after 34 days at 34 weeks of gestation. The neonate was 2260 grams and had no complications. Neither one of these two patients had histological evidence of chorioamnionitis. There were two patients with MR scores of 2 also representing apparent false negative results. Both presented with pre term PROM and amniotic fluid cultures were positive for Prevotella oralis or Gardenella vaginalls but without evidence of intra-amniotic inflammation. Both patients were induced and delivered within 5 days but the placentas had no evidence of acute chorioamnionitis at delivery. Thus, the apparent false negative cases of the proteomic analysis did not have any clinical or histological evidence of inflammation or complications of prematurity.

There were 5 patients with abnormal MR scores (3-4) and negative amniotic fluid cultures (false positive results for infection). All had amniotic fluid WBC counts over 100/mm³, evidence of histological chorioamnionitis and delivered preterm neonates shortly after admission. Thus all apparent false positive results were associated with abnormal outcomes and histological evidence of acute inflammation.

Survival analysis of the duration of pregnancy demonstrated that patients with an MR score of 3 or 4 had a shorter amniocentesis to delivery interval than those with an MR score of 0 to 2 (log rank Mantel-Haenszel test Chi square 42.6, p<0.0001). The median amniocentesis to delivery interval was 1 day in patients with MR scores of 3 or 4 but 19 days for patients with MR scores of 0, 1 or 2 (95% Cl of ratio: 18-20 days) (FIG. 3b). The difference remained highly significant when 26 patients delivered for maternal or foetal-indications were censored (median amniocentesis to delivery interval for patients with MR scores of 0-2=43 days vs. 2 days for MR scores of 3-4; log rank Chi-square 22.7, p<0.0001, ratio: 21.5 days, 95% Cl of ratio: 21-22).

(2) Test of the MR Score in a Different Population of Preterm Patients

We examined the performance of the MR score by analyzing blindly a separate set of 24 samples of amniotic fluid. Two investigators selected from the fluid bank 12 “diseased” and 12 “non-diseased” samples. The selection criteria for the diseased patients were: 1) diagnosis of preterm labor with intact membranes; 2) intra-amniotic inflammation (amniotic fluid WBC>100/mm³) and acute histological chorioamnionitis; 3) spontaneous preterm delivery shortly after amniocentesis. This group consisted of two subgroups of patients: 6 with a positive amniotic fluid culture for microorganisms and 6 with a negative amniotic fluid culture. Control patients were with the diagnosis of preterm labor and intact membranes, before 33 weeks, had no elevated WBC In amniotic fluid, negative cultures and delivered at term. The selection criteria were chosen to test the profile in patients with either clearly defined disease or absence of disease as In premature labour and in contrast to other conditions (e.g., cancer) no real “gold standard” for disease is available to classify patients at the time of amniotic fluid analysis. Samples were randomly coded and two other investigators performed the SELDI experiments as described and independently analyzed and scored the spectra. The MR scores of 3 or 4 diagnosed with 100% sensitivity and specificity the presence of intra-amniotic inflammation. In addition, by quick visual evaluation of the presence or absence of the peaks composing the MR score on the CHCA-LL and SPA SELDI protocols relative to the R peak present in all samples, the investigators were able to subjectively diagnose inflammation again with 100% sensitivity and specificity. There was 100% inter-rater agreement (kappa=1) on scores and diagnosis. The method could not discriminate the group with microbiologically proven intra-amniotic infection from that with intra-amniotic inflammation and negative amniotic fluid culture. The proteomic profile therefore, identifies intra-amniotic inflammation rather than Infection.

(3) Identity of Biomarkers Composing the MR Score

We reasoned that P2 and P1 might be the neutrophil defensins 1 and 2 (HNP-1 and -2, respectively), based upon the distinctive peaks of these antimicrobial peptides previously detected by SELDI-TOF in crevicular fluid from patients with periodontitis. To determine whether P1 and P2 correspond to neutrophil defensins, we proceeded with an on-chip immunoassay, using a polyclonal antibody, that does not distinguish between the three HNP peptides as each differ just by one amino acid.

FIG. 4a illustrates that the P1, P2 and P3 peaks present in profiling spectra (on H4 spots) also are detected on the spots pre-coated with the anti-HNP-1-3 antibody but not the IgG coated spots. In addition, the presence of the peptides was confirmed in samples from “diseased” patients at the appropriate mass on Comassie stained gels (FIG. 4b) and by western blotting (FIG. 4a-insert).

We estimated the accuracy of mass determination by comparing the average masses of P1 and P2 in amniotic fluid with the average masses of the peaks obtained by spotting recombinant HNP-1 and HNP-2 peptides on triplicate spots of a mixture of equal amounts (0.5 ng each). The observed (in amniotic fluid) and calculated (SwissProt) masses for HNP-1 and 2 are listed in Table II. The intra-assay variability calculated from the triplicate spots with recombinant peptides was 0.07% and the mass accuracy in both simple (mixture of recombinant peptides) and complex (amniotic fluid) samples was 0.2%.

TABLE II
Biomarkers composing the M and MR scores

| | observed mass [95% CI]; | calculated | |
|---|---|---|---|
| Biomarker | PI | mass; PI | Protein ID |
| [symbol] | 3378.2 [3377.0-3379.4]; | 3377.01; | HNP-2 |
| | PI > 8 | 8.67 | |
| [symbol] | 3449.7 [3448.5-3451.0]; | 3448.09; | HNP-1 |
| | PI > 8 | 8.68 | |
| P3 | 3493.0 [3491.4-3494.5] | | |
| P4 | 3720.7 [3719.5-3721.9] | | |
| P5 | 4149.0 [4147.5-4150.4] | | |
| P6 | 4629.6 [4603.5-4655.7] | | |
| [symbol] | 10471.7 [10467.9-10475.5]; | 10443.85; | Calgranulin C |
| | PI: 4-6 | 5.82 | |
| [symbol] | 10874.4 [10868.6-10880.2]; | 10834.51; | Calgranulin A |
| | PI: 4-6 | 6.51 | |
| P9 | 11362.3 [11355.2-11369.3] | | |
| R peak | 11790.4 [11785.9-11794.9] | | |
| P10 | 12221.0 [12213.8-12228.1] | | |
| P11 | 12354.4 [12345.3-12363.5] | | |
| P12 | 12730.5 [12724.5-12736-4] | | |
| P13 | 13329.2 [13323.2-13335.3] | | |

Circled peaks are components of the MR score;
PI: isoelectric point;
R peak: reference peak

To establish the identity of P7 and P8, first we performed in-gel tryptic digest as described above. Concurrently, the exact mass of the band to be digested was determined, by eluting the intact protein from pieces of the excised gel and subjecting it to miss spectrometry. This technique confirmed that an excised gel band corresponded indeed to the peak of interest on SELDI.

The observed mass of the intact protein, isoelectric point (Table 11), and tryptic peptide masses were entered in searchable databases:

ProFound (http://129.85.19.192/profound bin/WebProFound.exe), Peptldent (http:www.expasy.ch/tools/peptident.html), and MS-Fit (http://prospector.ucsf.edu/ucsfhtm14.0/msfit.htm). We matched P7 to calgranulin C (accession #P805111) and P8 to calgranulin A (accession #P05109), after which we proceeded with an on-chip immunoassay, using a monoclonal antibody that recognized calgranulins A, B and C (1:1000, Mac 387) (FIG. 4c). FIGS. 4b and 4d show the bands of Interest on a Comassie stained gel and conventional western blotting, using the same antibody used for the on-chip immunoassay.

We attempted to determine whether a rapid SELDI analysis of amniotic fluid, pursuant to the invention, also could provide quantitative information about the degree of intra-amniotic inflammation, a significant prognostic indicator of fetal outcome. This information currently is unavailable to practitioners except for the WBC count of amniotic fluid and clinical symptoms (maternal fever, uterine tenderness or fetal tachycardia) as the rest of the rapid tests (e.g., Gram stain) diagnose infection rather than inflammation.

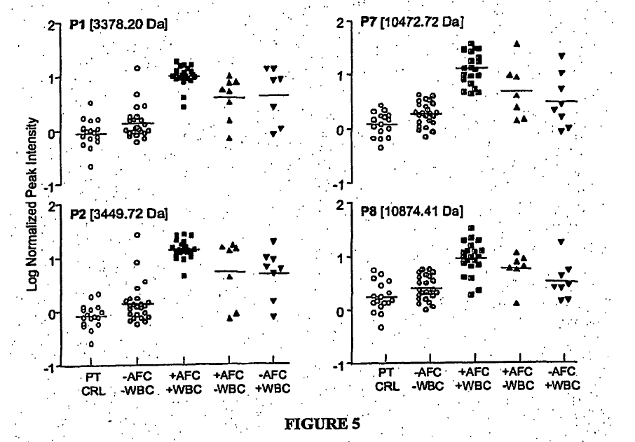

For this purpose, we analyzed the log-normalized intensity of the MR peaks from the 77 preterm patients studied in the “learning” phase. After logarithmic transformation, all data sets of peak Intensities were normally distributed. As expected, there was a significant difference in the mean signal intensity of all 4 peaks between the extreme “non-diseased” and “diseased” groups (p<0.001) (FIG. 5). There was also a significant difference in peak intensities between patients with preterm labor who delivered at term (PT-CRL) and the group without infection or elevated WBC count that delivered preterm (−AFC-WBC, in Table I and FIG. 5), suggesting that at least in some of the patients delivering preterm, there is evidence of intra-amniotic inflammation even with WBC counts <100/mm³.

(4) SELDI Profile of Amniotic Fluid at Term.

Five of the 13 samples of amniotic fluid at term had detectable P1 and P2 peaks on SELDI (i.e., above the S/N cut-off) (FIG. 2a). However, none showed visible P7 or PS peaks (FIG. 2c), and they therefore were diagnosed as “non-diseased” by MR. This observation is consistent with increased availability of HNPs in amniotic fluid with advancing gestational age. In addition, it suggests dissociation between the presence of neutrophil defensins (HNP-1 and -2) and calgranulins in amniotic fluid, and it emphasizes the value of MR score as a composite diagnostic test.

HNP-1-3 are peptides with antimicrobial activity involved in innate immunity and are present in high amounts in azurophilic granules of activated neutrophils. Accordingly, we, reasoned that the intensity of the HNP peaks (P1 and P2) should reflect the degree of neutrophil activation in the amniotic cavity, and since these cells are thought to be of foetal origin the extent of fetal inflammation. To test this hypothesis, we first investigated our ability by SELDI to quantitate mixtures of recombinant HNP-1 and -2 peptides reliably.

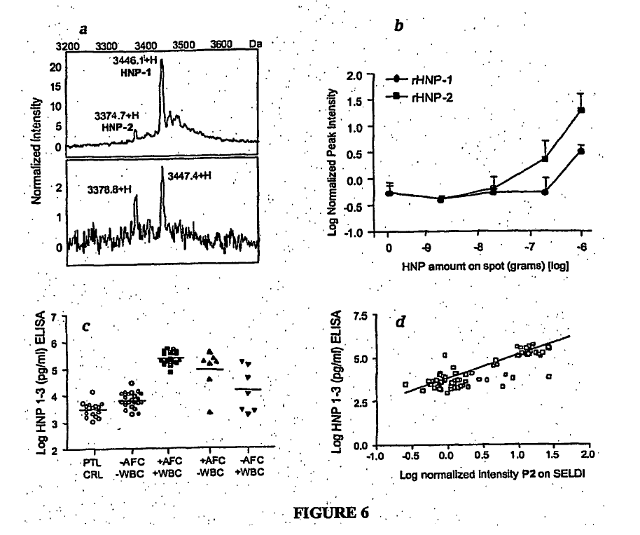

Between 20 ng and 1 μg (peptide amount on-chip) there was a dose-dependent relationship with the normalized peak intensity (FIGS. 6a and 6b). The limit of detection for HNP-1 was 5.8 fmols. Interestingly, in the presence of HNP-1, the intensity of the HNP-2 peak was significantly lower, suggesting either a peak suppression effect or differences in ionizing abilities in the mass spectrometer between the two peptides (FIG. 6a). These results identified HNP-1 (P2) as a better quantitative indicator of intra-amniotic inflammation than HNP-2 (P1).

To examine the accuracy of our quantitative estimation in a complex sample such as amniotic fluid, we also measured the concentration of peptides by ELISA. This assay quantifies the total HNP-1-3 amount since the antibody used recognizes a common epitope on the three peptides. ELISA confirmed the significant differences in HNP 1-3′ concentrations among groups seen with SELDI (FIG. 6c). There was a significantly linear correlation between the log of the HNP amount measured by ELISA and the log of the normalized peak Intensity (r²=0.701 for P1, 0.703 for P2) (FIG. 6d). The correlation between HNP-1-3 estimated by SELDI and amniotic fluid white blood cell count (log WBC/mm³) was weaker (r²=0.491 for P1, 0.501 for, P2) than that observed between SELDI and HNP-1-3 by ELISA.

1-44. (canceled)

45. A diagnostic assay for detecting the presence of at least one biomarker indicative of intra-amniotic inflammation in a sample of amniotic fluid, comprising (A) mixing an adsorbent that binds at least one biomarker associated with intra-amniotic inflammation with a sample of amniotic fluid and then (B) monitoring said mixture for binding between said biomarker and said adsorbent, wherein said assay detects at least one biomarker that is a calgranulin.

46. A diagnostic assay as claimed in claim 45, wherein said adsorbent is an antibody immobilized on a solid substrate.

47. A diagnostic assay as claimed in claim 46, which is an ELISA.

48. A diagnostic assay as claimed in claim 46, wherein said solid substrate is a probe.

49. A diagnostic assay as claimed in claim 48, wherein said biomarker is detected by laser desorption/ionization mass spectrometry.

50. A diagnostic assay as claimed in claim 45, wherein said adsorbent is immobilized on a probe.

51. A diagnostic assay as claimed in claim 50, wherein said adsorbent is a hydrophobic adsorbent.

52. A diagnostic assay as claimed in claim 51, wherein said probe is a Ciphergen H4 probe or H50 probe.

53. A diagnostic assay as claimed in claim 45, which additionally tests for the presence of at least one defensin in said sample of amniotic fluid.

54. A diagnostic assay as claimed in claim 53, wherein said defensin is HNP-1 (alpha-defensin 1).

55. A diagnostic assay as claimed in claim 47, which additionally tests for the presence of at least defensin in said sample of amniotic fluid.

56. A diagnostic assay as claimed in claim 55, wherein said defensin is HNP-1 (alpha-defensin 1).

57. A diagnostic assay as claimed in claim 50, which additionally tests for the presence of at least defensin in said sample of amniotic fluid.

58. A diagnostic assay as claimed in claim 57, wherein said defensin is HNP-1 (alpha-defensin 1).

59. A diagnostic assay as claimed in claim 45, wherein said calgranulin is calgranulin A.

60. A diagnostic assay as claimed in claim 45, wherein said calgranulin is calgranulin C.

61. A kit for detecting the presence of at least one biomarker indicative of intra-amniotic inflammation in a sample of amniotic fluid, comprising:
at least one adsorbent that binds at least one biomarker associated with intra-amniotic inflammation; and
instructions for mixing said adsorbent with a sample of amniotic fluid and monitoring said mixture for binding between said adsorbent and a biomarker in said sample,
wherein said kit includes at least one adsorbent that detects a calgranulin.

62. A kit as claimed in claim 61, wherein said adsorbent is an, antibody is immobilized on a solid substrate.

63. A kit as claimed in claim 62, which additionally comprises an enzyme-antibody conjugate used to detect biomarker immobilized on said solid substrate.

64. A kit as claimed in claim 61, wherein said solid substrate is a probe.

65. A kit as claimed in claim 64, wherein said kit instructions specify analysis by laser desorption/ionization mass spectrometry.

66. A kit as claimed in claim 62, wherein said solid substrate is a probe.

67. A kit as claimed in claim 66, wherein said adsorbent is a hydrophobic adsorbent.

68. A kit as claimed in claim 67, wherein said probe is a Ciphergen H4 probe or H50 probe.

69. A kit as claimed in claim 61, additionally comprising at least one adsorbent that binds to at least one defensin.

70. A kit as claimed in claim 69, wherein said defensin is HNP-1 (alpha-defensin 1).

71. A kit as claimed in claim 63, which additionally comprising at least one adsorbent that binds to at least one defensin.

72. A kit as claimed in claim 71, wherein said defensin is HNP-1.

73. A kit as claimed in claim 65, which additionally comprising at least one adsorbent that binds to a defensin.

74. A kit as claimed in claim 73, wherein said defensin is HNP-1 (alpha-defensin 1).

75. A kit as claimed in claim 61, wherein said calgranulin is calgranulin A.

76. A kit as claimed in claim 61, wherein said calgranulin is calgranulin C.

77. A method for qualifying the risk of preterm delivery in a pregnant patient, comprised of analyzing a sample of amniotic fluid from said patient for a level of at least one calgranulin.

78. A method according to claim 77, additionally comprising analyzing said sample for the level of at least one defensin.

79. A method according to claim 77, wherein said calgranulin is calgranulin A or calgranulin C.

80. A method according to claim 78, wherein said defensin is HNP-1 (alpha-defensin 1) or HNP-2 (alpha-defensin 2).

81. A method according to claim 80, wherein said defensin is HNP-1 (alpha-defensin 1).

82. A method for qualifying the risk of preterm delivery in a pregnant patient, comprising
(A) providing a spectrum generated by subjecting a sample of amniotic fluid from said patient to mass spectroscopic analysis that includes profiling on a biologically- or chemically-derivatized affinity surface and
(B) putting said spectrum through pattern-recognition analysis that is keyed to at least one peak indicative of the presence of a calgranulin in said sample.

83. A method according to claim 82, wherein said pattern-recognition analysis additionally is keyed to at least one peak indicative of a defensin.

84. A method according to claim 82, wherein said calgranulin is calgranulin A or calgranulin C.

85. A method according to claim 83, wherein said defensin is HNP-1 (alpha-defensin 1) or HNP-2 (alpha-defensin 2).

86. A method according to claim 85, wherein said defensin is HNP-1 (apha-defensin 1).

87. A method according to claim 85, wherein said calgranulin is calgranulin A or calgranulin C.

88. A method according to claim 86, wherein said calgranulin is calgranulin A or calgranulin C.

89. A method according to claim 82, wherein said chemically-derivatized affinity surface is a Ciphergen H4 probe or H50 probe.

90. A method according to claim 82, wherein said patient does not have a white blood cell count that is elevated out of the normal range.
